(19) United States
(12) Patent Application Publication
(10) Pub. No.: US 2004/0022842 A1
(43) Pub. Date: Feb. 5, 2004

(54) Liposome preparations containing oxaliplatin

(75) Inventors: Masazumi Eriguchi, Tokyo (JP); Hironobu Yanagie, Tokyo (JP); Kazuo Maruyama, Kanagawa (JP); Tadashi Fujisawa, Tokyo (JP)

Correspondence Address: FOLEY & LARDNER, P.O. BOX 80278, SAN DIEGO, CA, 92138-0278, US

(73) Assignee: Mebiopharm Co., Ltd.

(21) Appl. No.: 10/454,392

(22) Filed: Jun. 3, 2003

Related U.S. Application Data
(63) Continuation-in-part of application No. 10/270,261, filed on Oct. 11, 2002.

(30) Foreign Application Priority Data
Jun. 3, 2002 (JP) 2002-161296

(51) Int. Cl. A61K 39/395; A61K 9/127; A61K 31/728

(52) U.S. Cl. 424/450; 424/178.1; 514/54

(57) ABSTRACT

The present invention provides a liposome preparation containing oxaliplatin and derivatized with a hydrophilic polymer and a ligand. In one embodiment the hydrophilic polymer is polyethylene glycol and the ligand is transferrin. In accordance with the invention, the uptake of a pharmaceutical agent contained in the liposome into tumor cells can be enhanced through transferrin receptors expressed on the surface of the tumor cells. Also provided are pharmaceutical compositions containing the liposomes and methods of their use.

The present application is a continuation-in-part of U.S. application Ser. No. 10/270,261, filed Oct. 11, 2002, and claims the benefit of Japanese patent application No. 2002-161296, filed Jun. 3, 2002, which are hereby incorporated by reference in their entireties, including all tables, figures, and claims.

### FIELD OF THE INVENTION

The present invention relates to a liposome preparation for use as an anti-tumor agent.

### BACKGROUND OF THE INVENTION

The following discussion of the background of the invention is merely provided to aid the reader in understanding the invention and is not admitted to describe or constitute prior art to the present invention.

Cisplatin has been widely used as an anti-tumor agent for the treatment of various cancers including testis tumor, bladder tumor, renal pelvis and ureter tumor, prostate cancer, ovarian cancer, head and neck cancer, non-small cell lung cancer, esophageal cancer, cervical cancer, neuroblastoma and gastric cancer. However, cisplatin has disadvantages in that it is highly toxic and is usually associated with adverse side effects such as renal disorders including acute renal failure, inhibition of the bone marrow function, nausea, vomiting and anorexia. For the purpose of overcoming these disadvantages, cisplatin derivatives such as carboplatin and oxaliplatin have been developed. Oxaliplatin exerts therapeutic activities similar to those of cisplatin and has relatively low nephrotoxicity and emetogenicity.

Liposomes are sometimes used in an effort to reduce the toxicity of certain agents. However, many compounds cannot be effectively encapsulated in liposomes, and additional problems arise with the stability of liposome formulations. The liposome formulation must also be delivered efficiently to the target cells.

Accordingly, compositions and methods that improve the storage stability of liposome preparations containing anti-tumor agents and the efficiency of their delivery to target tumor cells would be of great value.

### SUMMARY OF THE INVENTION

The present invention provides a formulation of liposomes containing oxaliplatin contained within the liposomes. The liposomes are derivatized with a hydrophilic polymer and a ligand. In various embodiments the ligand is selected from the group consisting of transferrin, folic acid, hyaluronic acid, a sugar chain such as galactose or mannose, a monoclonal antibody, pyridoxal phosphate, vitamin B12, sialyl Lewis X, epidermal growth factor, basic fibroblast growth factor, vascular endothelial growth factor, vascular cell adhesion molecule (VCAM-1), intercellular adhesion molecule (ICAM-1), platelet endothelial adhesion molecule (PECAM-1), an Arg-Gly-Asp (RGD) peptide, or an Asp-Gly-Arg (NGR) peptide, and a Fab′ fragment of a monoclonal antibody. In various embodiments the hydrophilic polymer is selected from the group consisting of polyethylene glycol (PEG), polymethylethylene glycol, polyhydroxypropylene glycol, polypropylene glycol, polymethylpropylene glycol, and polyhydroxypropylene oxide. In one embodiment the hydrophilic polymer is polyethylene glycol and the ligand is transferrin.

“Polymers” are composed of two or more smaller molecules (monomer) covalently bonded together. “Hydrophilic polymers” are polymers that are generally soluble in aqueous solutions. In various embodiments the liposome preparation contains oxaliplatin at a concentration of 1-20 mg/ml or 1-10 mg/ml, or 5-10 mg/ml, or 10-15 mg/ml, or 15-20 mg/ml, or at about 8 mg/ml, or at 7.5-8.5 mg/ml, or at 7-9 mg/ml or 6-10 mg/ml or 7-10 mg/ml. In this context “about” means plus or minus 10%. In other embodiments any concentration of oxaliplatin can be used such as, for example, about 5%, 6%, 7%, 8%, 9%, 10%, 11%, 12%, 13%, 14%, 15%, 16%, 17%, 18%, 19%, 20%, 21%, 22%, 23%, 24%, or 25% (w/v) . In one embodiment the liposome formulation is provided in a sucrose solution. In various embodiments the concentration of sucrose in the solution is about 5% or about 6%, about 7%, about 8%, about 9%, about 10%, about 11%, about 12% about 13%, about 14%, or about 15% (w/v). In various embodiments the solution is about 9% (w/v) sucrose, or 8.5-9.5% (w/v) sucrose, or 8-10% (w/v) sucrose, or 7-11% (w/v) sucrose. By “derivatized” is meant that the hydrophilic polymer and ligand are covalently associated with the liposome. Derivatization can occur by attaching the ligand to a hydrophilic polymer or other molecule that is attached to a molecule that is stably retained in the lipid bilayer of the liposome. For example, the ligand can be attached to a distal end of a hydrophilic polymer, and a proximal end of the hydrophilic polymer is attached to the polar head group of a phospholipid stably retained in the bilayer of the liposome. Derivatization can also occur by attachment of the ligand to a molecule that is itself stably retained within the lipid bilayer of the liposome. For example, the ligand can be attached directly to a phospholipid member of the liposome bilayer. In another embodiment derivatization can occur by covalently attaching the ligand to a linker which is attached to a hydrophilic polymer, which itself is covalently attached to a molecule embedded in the lipid bilayer of the liposome, or by attaching the ligand to a linker that is itself attached to a molecule embedded in the lipid bilayer of the liposome.

In one embodiment derivatization occurs by covalently attaching a hydrophilic polymer to a fat soluble molecule (e.g., a phospholipid or fatty acid), which is a part of the bilayer of the liposome, and covalently attaching the ligand to the hydrophilic polymer (directly or through a linker). In one embodiment the hydrophilic polymer is covalently attached at one end to the head group of the lipid in the liposome (e.g., a PEG molecule is covalently attached to a distearoylphosphatidylethanolamine—DSPE) and attached to the ligand at the other end. A “ligand” refers to a substance that binds to a receptor or surface antigen located on the surface of a mammalian cell. By “sugar chain” is meant monosaccharides, disaccharides, oligosaccharides, and polysaccharides. In various embodiments the sugar chain is a galactose or mannose molecule, or a polymer thereof. But in other embodiments the sugar chain is a rhamnose, fucose, xylose, arabinose, or glucose molecule, or a chain of two or more of any of these or other monosaccharides. In still other embodiments the sugar chain is a disaccharide, such as sucrose, lactose, maltose, isomaltose, trehalose, cellobiose, or polymers of any of these, or an oligosaccharide or polysaccharide chain.

In another aspect the present invention provides a liposome formulation containing oxaliplatin contained in a liposome. A hydrophilic polymer is attached to a phospholipid that is stably retained within the bilayer of the liposome, and a ligand is attached to the hydrophilic polymer. By “stably retained” is meant that the polymer is associated with the liposome such that at least 75% of the polymers are still associated with the liposome after 90 days of storage at a temperature of 4° C. In various other embodiments at least 75% or 80% or 85% or 90% or 95% or 98% of the polymer is still associated with the liposome after 30 days, or 60 days, or 90 days, or 120 days, or 150 days, or 180 days of storage at 4° C. In one embodiment the polymer is stably retained by being covalently attached (either directly or indirectly through a linker) to a phospholipid that is a part of the liposome bilayer.

In another aspect the present invention provides pharmaceutical compositions for the treatment of tumors. The pharmaceutical composition includes a formulation of the invention and a pharmaceutically acceptable carrier.

The present invention also provides methods of treating tumors. The methods involve administering to a patient in need of such treatment a formulation or pharmaceutical composition of the present invention. Persons in need of such treatment include persons having a tumor or having cancer, but also includes persons where the development of a tumor is sought to be prevented or arrested. The composition can be administered to either shrink or destroy an existing tumor, but also to prevent an existing tumor from becoming larger or more pervasive. In various embodiments the tumor is a colorectal cancer, a gastric cancer, a hepatic cancer, a lung cancer, a breast cancer, an ovarian cancer, a pancreatic cancer, an esophogeal cancer, or another type of cancer.

The summary of the invention described above is not limiting and other features and advantages of the invention will be apparent from the following detailed description of the preferred embodiments, as well as from the claims.

### BRIEF DESCRIPTION OF THE DRAWINGS

FIG. 1 is a schematic representation showing the production process for a transferrin-conjugated liposome according to the present invention.

FIG. 2 is a graph showing the cytotoxicity of oxaliplatin. The y-axis represents the number of L-OHP treated cells that survive after treatment with 1 ug/ml, 5 ug/ml, or 100 ug/ml of L-OHP. The value is expressed as the % of the number of untreated control cells (i.e., at an L-OHP concentration of zero). The data illustrate that the LD₅₀ (i.e., the concentration where 50% of cells will die) is 8 ug/ml.

FIG. 3 is a table showing physical properties of a unmodified liposome, a PEG liposome, and a Transferrin-PEG liposome.

FIG. 4 is a graph showing the number of transferrin receptors present on the cell surface in normal leukocytes and in various types of tumor-derived cell lines.

FIG. 5 is a table showing the occurrence of bloody ascites and tumor nodules in mice administered with an unmodified liposome, a PEG liposome, and a Transferrin-PEG liposome.

FIG. 6 is a graphical illustration showing the cytotoxicity of oxaliplatin liposomes of the invention against Colon 26 cells. The illustration shows that the LD₅₀ values of oxaliplatin (L-OHP) solution, a Bare-liposome formulation, PEG-liposomes, and TF-PEG-liposomes on Colon 26 cells are 2 μg/mL, 60 μg/mL, 18 μg/mL and 8 μg/mL, respectively.

FIG. 7 is a graphical illustration showing the cytoxocity of oxaliplatin liposomes of the invention against AsPC-1 cells. The illustration shows that the LD₅₀ values of oxaliplatin (L-OHP) solution, a Bare-liposome formulation, PEG-liposomes, and TF-PEG-liposomes on AsPC-1 cells are 5 μg/mL, 45 μg/mL, 75 μg/mL and 8 μg/mL, respectively.

### DETAILED DESCRIPTION OF THE INVENTION

Liposomes or lipid vesicles are spherical lipid bilayers having an inner aqueous core onion-like structures comprising a series of bimolecular lipid layers spaced from one another by an aqueous solution, the outermost layer being lipid. Liposomes have been advantageously used to encapsulate biologically active materials for a variety of uses. Liposomes can be unilamellar or multilamellar. Multilamellar liposomes are composed of a number of bimolecular lamellae interspersed with an aqueous medium. They have onion-like structure containing a series of bimolecular lipid layers spaced from one another by an aqueous solution with the outermost layer being lipid. Unilamellar vesicles have a single spherical lipid bilayer entrapping aqueous solution. According to their size they are referred to as small unilamellar vesicles (SUV) with a diameter of 250 nm or less, and large unilamellar vesicles (LUV) with a diameter of greater than 250 nm. Either unilamellar (small or large) or multilamellar liposomes can be used in the present invention. In one embodiment of the invention the liposomes are small unilamellar vesicles and have a diameter of less than 200 nm.

During the formation of a liposome, molecules in an aqueous solution are entrapped in the aqueous center of the liposome and are thereby protected against the external environment. Liposomes injected in vivo are transported efficiently into the cytoplasm of cells upon fusion of the liposome with the cell membrane.

Oxaliplatin, a platinum (II) cis-oxalato complex of trans-1-1,2-diaminocyclohexane, is a platinum complex compound represented by the following formula:

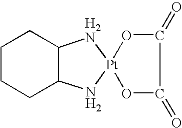

Oxaliplatin is useful as an anti-tumor or anti-cancer agent, since it has therapeutic activities similar to those of cisplatin but has relatively low nephrotoxicity and emetogenicity. It is particularly used in the treatment of colorectal cancer, but is also effective in the treatment of gastric cancer, hepatic cancer, lung cancer, breast cancer, ovarian cancer, pancreatic cancer, esophogeal cancer, and other cancers. The production process for oxaliplatin is well known in the art (see, for example, Japanese Patent Public Disclosure No. 9-40685). In the compositions according to the present invention, the oxaliplatin can be contained in an aqueous solution and the solution entrapped in the liposomes such that the oxaliplatin is present at a concentration of from 1 to 20 mg oxaliplatin/ml of solution in the liposome. In one embodiment the liposome preparation of the present invention contains from 1 to 20 μg of oxaliplatin per mg of lipid, and from 100 to 300 μg of ligand per mg of lipid.

In one embodiment the liposomes contain oxaliplatin in solution of 9% sucrose (w/v), 8-10% sucrose (w/v), 8.5-9.5% sucrose (w/v), or about 9% sucrose (w/v). This concentration of sucrose is found to result in a greater amount of oxaliplatin being encapsulated in the liposome. Since a 9% sucrose solution is isotonic, oxaliplatin-containing liposomes can be diluted in physiological saline, and are stable in circulating blood. It was discovered unexpectedly that such liposomes can be delivered to tumor site (lesion) without substantial leakage of oxaliplatin from the liposome. In addition, as sugar solutions generally function to protect liposome membranes, such a liposome formulation can be subjected to freeze-drying and stored for a long period of time without adverse effects.

### Liposome Preparation

In one embodiment, the liposome preparation of the invention is produced by dissolving a phospholipid in a suitable organic solvent, dispersing the resultant solution in an aqueous solution containing a therapeutic agent, and then performing ultrasonication or reverse phase evaporation of the resultant dispersion. Many phospholipids can be used in the present invention. For example, phosphatidylcholines, phosphatidylethanolamines, distearoylphophatidyl-ethanolamine, phosphatidylserines, phosphatidylinositols, lysophosphatidylcholines, phosphatidylglycerols. sphingomyelins or phosphatidic acid will all find use in the present invention. For the purpose of modifying the stability or permeability of the lipid membrane, an additional lipophilic component can be added such as, for example, cholesterol or another steroid, stearylamine, phosphatidic acid, dicetyl phosphate, tocopherol, or lanolin extracts.

In other embodiments the lipid vesicles of the present invention can be produced from phospholipids, neutral lipids, surfactants or any other related chemical compounds having similar amphiphilic properties. These materials can be classified according to the formula A-B where A is a hydrophilic, generally polar group, e.g., a carboxyl group, and B is a hydrophobic, i.e., lipophilic, non-polar group, e.g., a long chain aliphatic hydrocarbon group. From the foregoing, it should be appreciated that the composition of the lipid component can be substantially varied without significantly reducing encapsulation efficiency, and other lipids, in addition to those listed above, can be used as desired.

In addition, liposomes can be modified with a hydrophilic polymer to prevent the uptake of the liposomes by the cellular endothelial systems and to enhance the uptake of the liposomes into tumor tissues. Modification of the liposomes with a hydrophilic polymer prolongs the half-life of the liposomes in the blood. Many hydrophilic polymers may be used in the invention including, for example, polyethylene glycol, polymethylethylene glycol, polyhydroxypropylene glycol, polypropylene glycol, polymethylpropylene glycol, polyhydroxypropylene oxide, polyoxyalkylenes, polyetheramines. Additional polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, and polyaspartamide. The polymers may be employed as homopolymers or as block or random copolymers.

### Ligand Derivatization

The liposome formulations of the present invention are further characterized in that the liposomes are derivatized with a ligand. In one embodiment, the ligand is transferrin. Transferrin is an iron-binding protein that facilitates uptake of iron into mammalian cells by the endocytosis of transferrin-bound Fe³⁺via transferrin receptors located on the surface of cells. Transferrin thereby acts to supply iron to the cells. Transferrin receptors are generally expressed in tumor tissues in greater quantities compared with normal cells. This is true for many different types of tumors. Therefore, by binding a therapeutic agent to transferrin, absorption of the therapeutic agent by the tumor cells is enhanced through the transferrin receptor.

In another aspect, the present invention provides a pharmaceutical composition for the treatment of a tumor, which is a liposome preparation of the present invention and a pharmaceutically acceptable carrier. The pharmaceutically acceptable carrier can be, for example, sterile water, a buffer solution, or saline. The pharmaceutical composition may further comprise various salts, sugars, proteins, starch, gelatin, plant oils, and polyethylene glycol as desired. In various embodiments the liposome formulations of the present invention are administered systemically via intravenous injection or hepatic artery injection. But other modes of administration can also be used such as, for example, local injection to a tumor site, intravenous or intra-arterial injection, instillation to the exterior of the tumor, or parenterally via bolus injection or continuous injection. Multilamellar liposome vesicles can also be administered by local injection, or even in the form of an ointment. The dosage may vary depending on the route of administration, the severity of the condition, the age and condition of the patient to be treated, and the degree of side effects, but is generally within the range from about 10 to about 100 mg/m²/day. The pharmaceutical compositions of the present invention are useful in the treatment of tumors and cancers.

The disclosure of all patents and documents cited herein are hereby incorporated herein by reference in their entirety, including all tables, figures, and claims. The following examples further illustrate the present invention. The examples below are not limiting and are merely representative of some aspects and features of the present invention.

### EXAMPLE1

### Preparation of Liposomes

This example provides one general strategy for preparing the liposome compositions of the invention. Of course other strategies are available, which will be appreciated by those of ordinary skill in the art with reference to this disclosure. This example describes the embodiment of using reverse phase evaporation (REV) to manufacture the liposome formulations of the present invention (see, for example, U.S. Pat. No. 4,235,871 for further details).

In order to stably retain the hydrophilic polymer within the lipid bilayer, one can first prepare a phospholipid derivative of the hydrophilic polymer, and then use the phospholipid derivative together with a phospholipid and a lipid to prepare the liposome. The hydrophilic polymer is synthesized as a derivative in which a phospholipid moiety is chemically bound to the polymer. The phospholipid moiety of the derivative therefore serves to stably retain the derivative in the bilayer of the liposome. The phospholipid derivative of the hydrophilic polymer may be prepared in such a manner as described in, for example, U.S. Pat. No. 5,013,556. A hydrophilic polymer such as polyethylene glycol is treated with cyanuric acid in a basic organic solvent to activate one terminus of the hydrophilic polymer, and the resultant product is then reacted with a phospholipid such as phosphatidylethanol, thereby obtaining a phospholipid-derivative of the hydrophilic polymer. The other terminus of the hydrophilic polymer can have a functional group, such as a carboxyl or maleimide group, to which the ligand is attached.

Thus, a phospholipid (e.g., distearoyl phosphatidylcholine (DSPC) or distearoyl phosphatidyl-ethanolamine (DSPE)), a lipid (e.g., cholesterol) and a phospholipid derivative of the hydrophilic polymer (e.g., polyethylene glycol-phosphatidyl-ethanolamine) are mixed together and then dissolved in a suitable organic solvent. The phospholipid and the lipid are mixed at a ratio of between 2:1 and 1:1, although any ratio between 3:1 and 1:3 is suitable. In one embodiment, DSPE-PEG is used and has a molecular weight of about 2750, of which PEG accounts for about 2000. The phospholipid derivative of the hydrophilic polymer is mixed with about 5 mole percent of the total lipid, although ratios of at least about 1 mole percent, at least about 2%, at least about 3%, at least about 4%, at least about 5%, at least about 6%, at least about 7%, at least about 8%, at least about 9%, or at least about 10% mole percent of total lipid derivative of the hydrophilic polymer are all suitable. In other embodiments at least about 15 mole % of total lipid is used. These amounts will result in liposomes that can remain in the blood for a maximal amount of time. The resultant solution is mixed with a solution of oxaliplatin in an aqueous buffer. In another embodiment the ratio of DSPC:CH:DSPE-PEG=1:1:0.1 (molar ratio), and the ratio of DSPE/PEG is about a 5 mole percent of total lipid. The concentration of oxaliplatin in the aqueous solution is from about 5 to about 10 mg/ml (although any concentration from 1 to 20 mg/ml is also suitable). The solvent mixture is sonicated and then evaporated to remove the solvent. The liposomes thus prepared are size-fractionated to afford oxaliplatin-containing liposomes having a diameter of about 0.2 μm.

Subsequently, the ligand is attached to the liposomes. In this example transferrin is used as the ligand. Transferrin is commercially available in the form of a purified protein (Biocompare, Burlington, Calif.). For the attachment of transferrin to the liposomes, one can previously introduce an additional functional group to the hydrophilic polymer attached to the phospholipid derivative that is retained in the membrane of the liposome. Thus, a hydrophilic polymer which has a carboxyl or maleimide group introduced at one terminus is added to a phospholipid to form liposomes having carboxyl or maleimide groups on the outer surface.

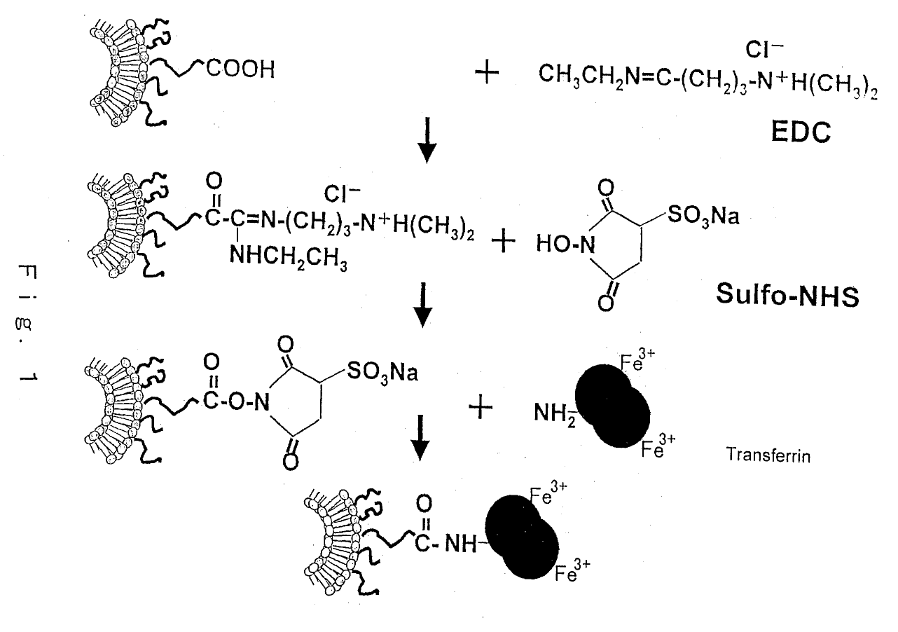

In the case where the terminus is a carboxyl group, 1-ethyl-3-(3-dimethylamino-propyl) carbodiimido hydrochloride (EDC-HCl) and N-hydroxysulfosuccineimide are bound to the liposomes. The resultant liposomes having the linkers attached are reacted with transferrin to obtain an apo-form of transferrin-bound liposomes in which transferrin is bound to the outer surface. The resultant liposomes are treated with iron citrate/sodium citrate to obtain the holo-form of transferrin-bound liposomes (FIG. 1).

In the case where the terminus has a maleimide group, the liposomes having the linkers attached are reacted with transferrin that has previously had an SH group introduced thereon, followed by the addition of iron in the same manner as described above to obtain the holo-form of trans ferrin-bound liposomes.

In addition to these examples, other amino, thiol, aldehyde and carbodiimide groups can be used as suitable linkers.

### EXAMPLE2

### Preparation of Lipsomes

In this alternative method of preparing liposomes, the lipid film forming step is conducted in a vessel partially filled with inert, solid contact masses. Significant variation is possible in the size, size distribution, shape and composition of the contact masses. The principal characteristics of the contact masses are: (1) that the contact masses be inert to the materials used in the formulation, in other words there should be no unwanted interaction between the contact masses and the lipid, lipophilic substances, organic solvent or aqueous liquid employed, and (2) that the contact masses be solid throughout the processing steps, in other words the contact masses should not dissolve or disintegrate and should provide an appropriate solid surface for supporting the thin lipid film. Prior experimental testing has used glass beads or balls as the inert, solid contact masses and these materials have proven to be particularly suitable. It is also expected that metal balls, e.g., stainless steel and synthetic substances, e.g., plastics, will also be suitable in appropriate circumstances. While spherical contact masses provide the maximum surface area in a given volume and are easily fluidized during the agitation step, other regular and irregular shapes could also be used.

The size of the contact masses used in any application will depend upon the scale of operation, the intensity of agitation, and other factors. As an example, it is normally appropriate to use contact masses having a size such that the ratio of the vessel volume to the volume of an individual contact mass is between 50 and 50,000. Generally, spherical contact masses will have a diameter between 1.0 mm and 100 mm. It is also contemplated that the contact masses could have a range or distribution of sizes. But equally sized contact masses adequately satisfy the requirements of the invention. The number of contact masses employed will depend upon their shape and size, the size of the vessel, the volume of organic solvent used, and the quantity of lipid and lipophilic substances dissolved. An appropriate number is used for increasing the surface area during the evaporation step and increasing the total area of the thin lipid film formed, but reserving sufficient volume within the vessel for movement of the contact masses during the agitation step.

The liposomes can be prepared by dissolving the lipid component (derivatized with hydrophilic polymer), together with any other lipophilic substances and the oxaliplatin in a suitable, generally non-polar, organic solvent. The organic solvent is selected so that it can be substantially removed from the lipid by evaporation and not otherwise affect any of the lipophilic substances included in the formulation. Examples of suitable solvents include ethers, esters, alcohols, ketones and various aromatic and aliphatic hydrocarbons, including fluorocarbons. The solvents may be used alone or in combination, for example, a 2:1 mixture of chloroform and methanol. The organic solvent is removed by evaporation, which can conveniently be accomplished by use of a rotary evaporator at temperatures generally between 20 C and 60 C and under less-than-atmospheric pressure. Evaporative conditions will depend upon the physical properties of the organic solvent and the lipophilic materials used in the formulation.

After the lipid film forming step, the lipids are hydrated with an aqueous liquid to form an aqueous dispersion of lipid. The required agitation can be accomplished by the rotation or translation, i.e., vibration, of the vessel. The presence of inert, solid contact masses within the vessel can provide an increased and consistent level of mechanical agitation, which enhances the formation of uniformly sized lipid vesicles. This hydration step is conducted above the transition temperature of the lipid components.

The aqueous liquid may be pure water; but can also be any aqueous solution of an electrolyte or a biologically active material. For example, an aqueous solution of sodium chloride or calcium chloride may be employed.

After agitating the lipid-aqueous liquid mixture, the resulting dispersion is allowed to remain undisturbed for a time sufficient to allow the lipid vesicles to form and mature. In one embodiment the vessel will stand undisturbed at room temperature for approximately one to two hours. The aqueous dispersion of the multilamellar lipid vesicles can then be recovered from the vessel containing the inert, solid contact masses. If desired, any non-incorporated active substances can be removed from the dispersion using known techniques such as repeated centrifugations, dialysis or column chromatography. The lipid vesicles can then be resuspended in any suitable electrolytic buffer for subsequent use.

At this point preparation of the liposomes is completed by attaching the linkers and ligand of choice, as described above.

### EXAMPLE3

### Cytotoxicity Test for Oxaliplatin

An oxaliplatin solution was prepared by dissolving oxaliplatin in a 9% sucrose solution at a concentration of 8 mg oxaliplatin/ml of solution.

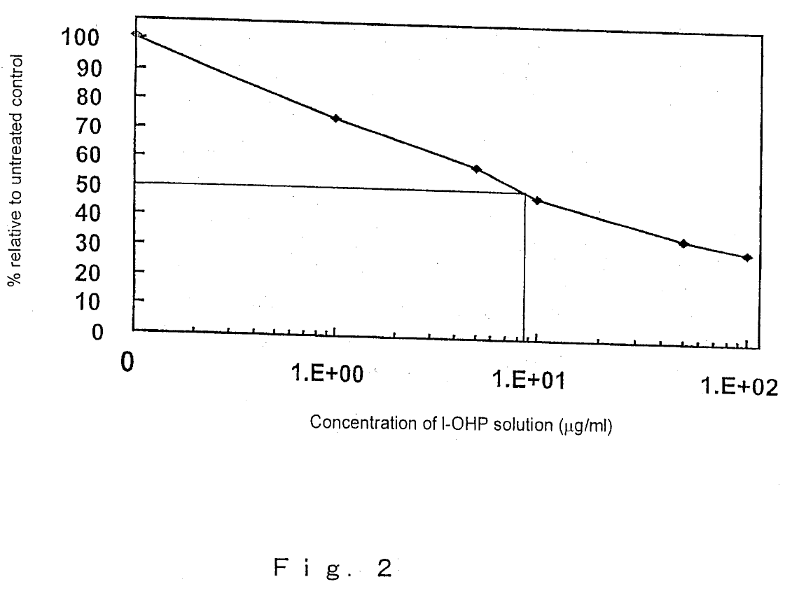

AsPC-1 cells were cultured in RPMI 11640 medium supplemented with 10% fetal calf serum with various concentrations of oxaliplatin solution at 37 C with 5% CO₂ for 4 hours. The medium was changed and the cells were cultured for an additional 48 hours. Cell viability was determined using a commercially available cytotoxicity assay kit (e.g., the APO-ALERT™ assay kit (Clontech, BD Biosciences Clontech, Palo Alto, Calif.). A substrate was added to the cells and incubated in 5% CO₂ for 2 hours. Color-development was measured at 450 nm (reference wavelength: 620 nm). The results are shown in FIG. 2. The oxaliplatin was found to have an LD₅₀>8 μg/ml.

### EXAMPLE 4

### Preparation of Oxaliplatin-Containing Liposome

This example provides another embodiment of the liposome compositions of the invention. The liposomes prepared according to this embodiment contain the following substances:

1. Distearoyl phosphatidylcholine (DSPC)

2. Cholesterol (CH)

3. N-(Carbamoylmethoxypolyethylene glycol 2000)-distearoyl phosphatidylethanloamine (DSPE-PEG-OMe)

4. Carboxyl polyethylene glycol 3000)-distearoyl phosphatidylethanolamine (DSPE-PEG-COOH)

These components were present at the following ratio: DSPC:CH:DSPE-PEG-OMe:DSPE-PEG-COOH=2: 1:0.19:0.01 (m/m). For the aqueous phase, an oxaliplatin solution (8 mg/ml, in a 9% sucrose solution) was used.

A mixture of DSPC, cholesterol, PEG2K-OMe, and PEG3K-COOH at the ratio of 2:1:0.19:0.01 (m/m) was dissolved in chloroform and isopropyl ether. The resultant solution was added with an oxaliplatin solution (in a 9% sucrose solution) and then sonicated. The solution was evaporated at 60° C. to remove the solvent and the lyophilization was repeated five times. The resultant product was sized at 60° C. using an EXTRUDER (Avestin, Ottawa, Canada) filter (twice at 400 nm and then five times at 100 nm), and then centrifuged twice at 200,000 g for 30 minutes. In the extruder process lipid vesicles are typically physically extruded under pressure through a polycarbonate filter that contains pores of pre-determined pore sizes. The resulting precipitate was resuspended in a 9% sucrose solution or 2-(N-Morpholino)-ethanesulfonic acid (MES) buffer (pH 5.5) to obtain oxaliplatin-PEG(-COOH/-OMe) liposomes.

The PEG-containing liposomes were the derivatized with transferrin (Tf). The oxaliplatin-PEG(-COOH/-OMe) liposomes prepared as above were then combined with 1-ethyl-3-(3-dimethylamino-propyl)carbodiimide hydrochloride (EDC) (in an amount of 2.7% relative to the weight of the lipid components) and N-hydroxysulfosuccineimide (S-NHS) (in an amount of 7.3% relative to the weight of the lipid components), and the mixture was allowed to stand at room temperature for 10 minutes. The resultant solution was reacted with transferrin (Tf) (in an amount of 20% relative to the weight of the lipid components) and then stirred at room temperature for 3 hours. The solution was centrifuged at 200,000 g for 30 minutes, and the precipitate was resuspended in a 9% sucrose solution.

The apo-form of Tf(-PEG) liposomes prepared as above were added with iron citrate-sodium citrate and then stirred at room temperature for 15 minutes. The resultant solution was centrifuged at 200,000 g for 30 minutes. The precipitate was resuspended in a 9% sucrose solution to obtain holo-form of Tf(-PEG) liposomes.

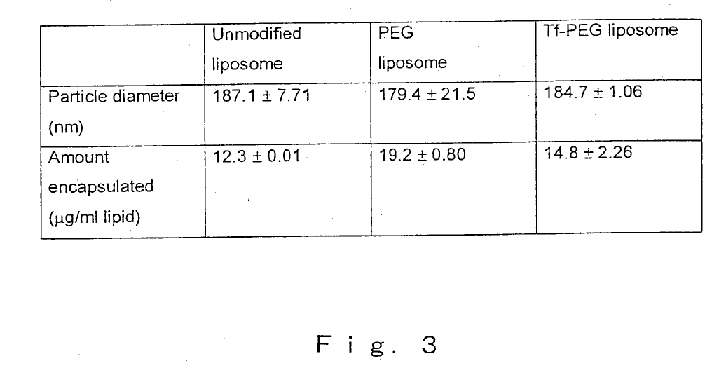

The physical properties of the unmodified liposome, PEG-liposome and Tf-PEG liposome prepared as above are summarized in FIG. 3.

### EXAMPLE5

### Determination of the Number of Transferrin Receptors on the Cell Surface

This example describes a method for determining the number of transferrin receptors on the surface of a cell. Normal human leukocytes and human cells of different malignant tumor-derived cell lines (K562, MKN45P and HL60) were used in this example. The number of transferrin (TF) receptors on the cell surface was determined by Scatchard analysis. A ¹²⁵I-labeled TF solution was added to a cell culture at different concentrations and incubated at 4° C. for 1 hour. The concentration of TF was determined by a protein determination assay and the radioactivity was measured using a gamma counter. The solution was centrifuged to precipitate the cells, and the cell fraction was washed with an ice-cooled buffer and then measured with a gamma counter to determine the concentration of TF bound to the cell surface. The number of cells was determined by a protein quantification assay. The concentration of unbound TF was determined by subtracting the concentration of bound TF from the known concentration of TF initially added. The number of bound TF (i.e., the number of the receptors) was determined from a Scatchard plot by plotting the concentration of bound TF on the vertical axis, and the ratio of the concentration of bound TF to the concentration of unbound TF on the horizontal axis. The number of bound TF (i.e., the number of the receptors) was determined from the x intercept of the graph.

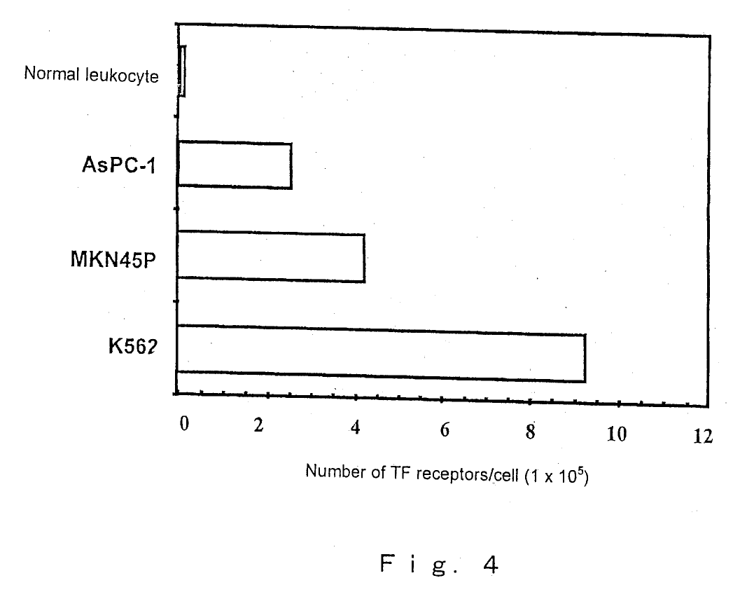

The amounts of ¹²⁵I-Tf bound to the cell surfaces in the different cell types are shown in FIG. 4. It was found that the number of transferrin receptors on the cell surfaces of the cell lines derived from the human malignant tumor was significantly higher than that in normal leukocytes.

### EXAMPLE6

### Therapeutic Effect of Oxaliplatin-Containing Liposome in Peritonea Inoculation Model

This example illustrates the therapeutic effectiveness of the liposome compositions of the present invention versus a simple oxaliplatin solution. Male BALB/c nu-nu nude mice aged 6 to 7 weeks were used as the animal models, and AsPC-1 cells (derived from human pancreatic cancer) and MKN45P cells (derived from human gastric cancer) were used as the tumor cells.

On day 0 of the experiment, AsPC-1 cells (2×10⁶ cells) or MKN45P cells (1×10⁷ cells) were intraperitoneally injected to the mice. On day 1 and day 4, liposomes prepared as described in Example 1 or oxaliplatin solution (8 mg/ml, in a 9% sucrose solution) was intraperitoneally injected into the respective mice. In both case, the concentration of oxaliplatin was adjusted to 5 mg oxaliplatin solution/kg body weight. Tf-PEG liposome, PEG liposomes, and unmodified liposomes were all used. PBS was administered as a negative control.

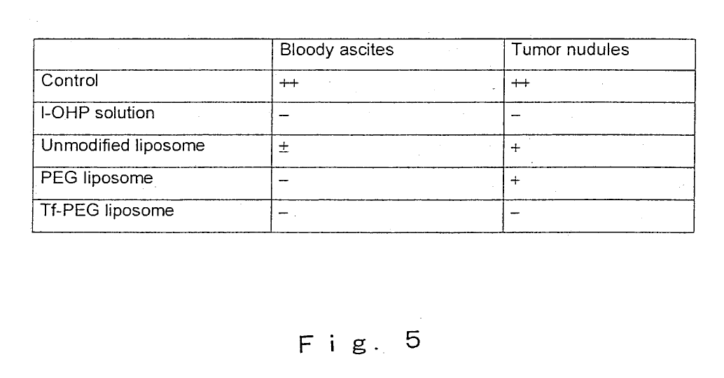

The AsPC-1 mice were trans-abdominally incised on day 21, and the MKN45P mice were trans-abdominally incised on day 16 and day 26. The presence or absence of bloody ascites and tumor nodules was noted. The results are shown in FIG. 5.

It was found that mice administered the Tf-PEG liposome of the present invention had significantly reduced occurrences of bloody ascites and tumor nodules compared to mice treated with unmodified liposomes or PEG liposomes.

### EXAMPLE7

### Cytotoxicity of Oxaliplatin (L-OHP) Liposomes Against Tumor Cells

Colon 26 cells and AsPC-1 cells were inoculated on a 96-well plate at 5×10³ cells per well and pre-incubated with 5% CO₂ overnight.

Oxaliplatin (L-OHP) solution or various types of liposomes were added to the cells at the concentrations indicated in Table 1. The cells were incubated at 37° C. under 5% CO₂ for 4 hours. The test solutions were removed, and the incubation was continued for an additional 2 days, at which time the number of living cells was counted. Cell viability was determined according to the WST-1 assay.

According to the WST-1 assay, the assay solution contained 5 mmol/L WST-1 reagent (10 μL); 20 mmol/L HEPES; 1-methoxy-PMS at 0.2 mmol/L, at pH 7.4. This solution was added to each well, mixed thoroughly, and incubated in a CO₂ incubator for 2 hours to allow for color development. The absorbance was then measured on a plate reader at 400-450 nm, with a reference wavelength of >600 nm.

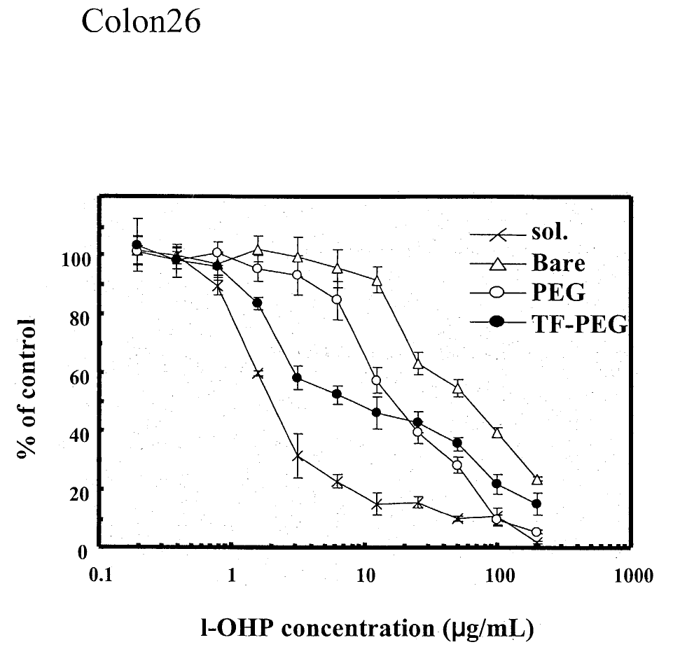

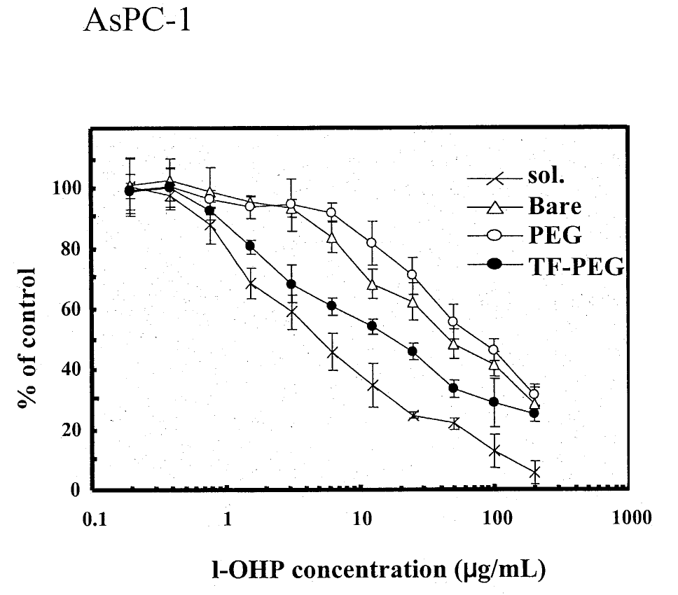

The cytotoxicity of oxaliplatin solution versus various types of liposome preparations on Colon 26 cells and AsPC-1 cells is illustrated in FIGS. 6 and 7, respectively. FIG. 6 shows that the LD₅₀ values of oxaliplatin (L-OHP) solution, Bare-liposomes, PEG-liposomes, and TF-PEG-liposomes on Colon 26 cells are 2 μg/mL, 60 μg/mL, 18 μg/mL and 8 μg/mL, respectively. The LD₅₀ values on AsPC-1 cells were 5 μg/mL, 45 μg/mL, 75 μg/mL and 8 μg/mL, respectively.

Therefore, L-OHP solution showed the highest toxicity. The ED₅₀ of TF-PEG-liposomes on Colon 26 cells was about 2.3-fold higher than that of the PEG-liposomes, and about 7.5-fold higher than Bare-liposomes. In AsPC-1 cells, the TF-PEG-liposomes showed about a 9.4-fold higher toxicity than PEG-liposome, and about a 5.6-fold higher toxicity than Bare-liposome. Additionally, the TF-PEG-liposomes showed cytotoxicity at a lower concentration than other liposome preparations.

TABLE 1

| | LD₅₀ | LD₅₀ for |
|---|---|---|
| | (Colo26 cells) | AsPC-1 cells |
| L-OHP solution | 2 μg/ml | 5 μg/ml |
| TF-PEG-Lipo encap. 1-OHP | 8 μg/ml | 8 μg/ml |
| PEG-Lipo encap. 1-OHP | 18 μg/ml | 75 μg/ml |
| Bare-Lipo encap. 1-OHP | 60 μg/ml | 45 μg/ml |

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

1. A liposome formulation comprising oxaliplatin comprised within the liposomes, and the liposomes derivatized with a hydrophilic polymer and a ligand.

2. A liposome formulation according to claim 1 wherein the ligand is selected from the group consisting of: transferrin, folic acid, hyaluronic acid, a sugar chain, a monoclonal antibody and a Fab′ fragment of a monoclonal antibody.

3. A liposome formulation according to claim 1 wherein the ligand is transferrin.

4. A liposome formulation according to claim 1 wherein the hydrophilic polymer is selected from the group consisting of polyethylene glycol, polymethylethylene glycol, polyhydroxypropylene glycol, polypropylene glycol, polymethylpropylene glycol and polyhydroxypropylene oxide.

5. The liposome formulation according to claim 1 wherein the hydrophilic polymer is polyethylene glycol.

6. A liposome formulation according to claim 1 wherein the oxaliplatin is present at a concentration of 6-10 mg/ml.

7. A liposome formulation according to claim 1 wherein the oxaliplatin is present at a concentration of 7-9 mg/ml.

8. A liposome formulation according to claim 1 wherein the oxaliplatin is present in an 8-10% sucrose solution.

9. A liposome formulation according to claim 1 wherein the oxaliplatin is present at a concentration of 7-9 mg/ml in an 8-10% sucrose solution.

10. A pharmaceutical composition for the treatment of tumor comprising a liposome preparation according to claim 6 and a pharmaceutically acceptable carrier.

11. A method for treatment of a tumor, comprising administering a patient in need of such a treatment a liposome preparation according to claim 6.

12. A liposome formulation comprising,
oxaliplatin comprised in a liposome, wherein a hydrophilic polymer is attached to a phospholipid that is stably retained within the bilayer of the liposome; and
wherein a ligand is attached to the hydrophilic polymer.

13. The liposome formulation of claim 12 wherein,
the hydrophilic polymer is selected from the group consisting of: polyethylene glycol, polymethylethylene glycol, polyhydroxypropylele glycol, polypropylene glycol, polymethylpropylene glycol and polyhydroxypropylene oxide; and
the ligand is selected from the group consisting of: transferrin, folic acid, hyaluronic acid, a sugar chain, a monoclonal antibody and a Fab′ fragment of a monoclonal antibody.

14. The liposome formulation according to claim 13 wherein the hydrophilic polymer is polyethylene glycol and the ligand is transferrin.

15. A liposome preparation according to claim 14 wherein the oxaliplatin is present in the liposome at a concentration of 7-9 mg/ml in a 8-10% sucrose solution.

16. A pharmaceutical composition for the treatment of tumor comprising
oxaliplatin comprised in a liposome, wherein a hydrophilic polymer is stably retained within the bilayer of the liposome and wherein a ligand is attached to the outer surface of the liposome; and
a pharmaceutically acceptable carrier.

17. The pharmaceutical composition of claim 16 wherein the hydrophilic polymer is selected from the group consisting of: polyethylene glycol, polymethylethylene glycol, polyhydroxypropylele glycol, polypropylene glycol, polymethylpropylene glycol and polyhydroxypropylene oxide; and
the ligand is selected from the group consisting of: transferrin, folic acid, hyaluronic acid, a sugar chain, a monoclonal antibody and a Fab′ fragment of a monoclonal antibody.

18. The pharmaceutical composition of claim 17 wherein the hydrophilic polymer is polyethylene glycol and the ligand is transferrin.

19. The pharmaceutical composition of claim 18 wherein the oxaliplatin is present in the liposome at a concentration of 7-9 mg/ml in a 8-10% sucrose solution.

20. A method for treatment of a tumor, comprising administering to a patient that has a tumor a liposome preparation comprising oxaliplatin comprised in a liposome, wherein a hydrophilic polymer is stably retained within the bilayer of the liposome and wherein a ligand is attached to the outer surface of the liposome; and
a pharmaceutically acceptable carrier.

21. The method of claim 20 wherein the hydrophilic polymer is selected from the group consisting of: polyethylene glycol, polymethylethylene glycol, polyhydroxypropylele glycol, polypropylene glycol, polymethylpropylene glycol and polyhydroxypropylene oxide; and
the ligand is selected from the group consisting of: transferrin, folic acid, hyaluronic acid, a sugar chain, a monoclonal antibody and a Fab′ fragment of a monoclonal antibody.

22. The method of claim 20 wherein the hydrophilic polymer is polyethylene glycol and the ligand is transferrin.

23. The method of claim 20 wherein the tumor is a pancreatic tumor or a gastric tumor.

24. The method of claim 20 wherein the tumor is a colorectal tumor.

25. The method of claim 20 wherein the oxaliplatin is present in the liposome at a concentration of 7-9 mg/ml in a 8-10% sucrose solution.
